(19) United States
(12) Patent Application Publication
(10) Pub. No.: US 2023/0190967 A1
(43) Pub. Date: Jun. 22, 2023

(54) Method and Composition for Evaluating Response to Neurodegenerative Disease Treatment Agent

(71) Applicant: GENUV INC., Seoul (KR)

(72) Inventors: Sungho Han, Seoul (KR); Mi-Yeon Kim, Seoul (KR); Yoon Sun Chun, Seoul (KR); Hyoung Tae Kim, Seoul (KR); Jina Park, Seoul (KR)

(73) Assignee: GENUV INC., Seoul (KR)

(21) Appl. No.: 17/965,675

(22) Filed: Oct. 13, 2022

Related U.S. Application Data
(63) Continuation of application No. PCT/KR2021/004662, filed on Apr. 13, 2021.

(30) Foreign Application Priority Data
Apr. 14, 2020 (KR) 10-2020-0045252

(51) Int. Cl. A61K 49/00; G01N 33/68; G01N 33/573; C12Q 1/6883; A61K 31/519; A61P 25/28

(52) U.S. Cl. CPC A61K 49/0004; G01N 33/6896; G01N 33/573; C12Q 1/6883; A61K 31/519; A61P 25/28; G01N 2800/52; G01N 2333/96466; C12Q 2600/158; C12Q 2600/106

(57) ABSTRACT

The present invention relates to: a method for evaluating response to treatment using a MEK 1/2 inhibitor in an individual diagnosed with a neurodegenerative disease; and a composition to be used for the method. Particularly, the method of the present invention comprises measuring, in a biological sample obtained from the individual with a neurodegenerative disease, the concentration of at least one biomarker selected from the group consisting of osteopontin, synaptotagmin-1, apolipoprotein-E, cathepsin B, HLA-DOB (HLA class II histocompatibility antigen, DO beta chain), and neurofilament light chain. According to the present invention, response to the MEK 1/2 inhibitor in the individual diagnosed with a neurodegenerative disease is monitored, and it is thereby possible to obtain useful information for managing the individual, such as determining the possibility of a treatment effect early on and determining whether to continue drug treatment and whether the amount needs to be adjusted.

### TECHNICAL FIELD

The present invention relates to a method for evaluating response to treatment with a MEK 1/2 inhibitor in a subject diagnosed with a neurodegenerative disease, and a composition for use in said method.

### BACKGROUND ART

Biomarkers are biological indicators that can distinguish normal or pathological states in a particular disease or predict or objectively measure treatment responses to drugs. According to their use, biomarkers can be classified into diagnostic biomarkers to detect and diagnose diseases, prognostic biomarkers to predict the likelihood of specific clinical events, disease recurrence or progression in patients, predictive biomarkers to predict therapeutic responses in patients, and pharmacodynamic/response biomarkers to show a biological response has occurred in patients who have been exposed to a therapeutic agent.

Especially in developing treatments for neurodegenerative diseases, such as Alzheimer's disease, which has a very low likelihood of success, there is a high demand for biomarkers, even from a clinical trial stage, to select test subjects, to prove that candidate drugs act on targets, to generate evidence that the disease has been fundamentally cured, to monitor side effects, and the like. Many studies are being conducted to increase the possibility of success in the development of treatments through the use of a biomarker.

Trametinib is a MEK 1/2 inhibitor, which inhibits both MEK1 and MEK2, the upstream components of ERK in the MAPK/ERK signal transduction pathway. It is commercially available under the tradename Mekinist® or Meqsel® and is used as a cancer drug for melanoma, non-small cell lung cancer, and the like. Recently, trametinib was presented as a therapeutic candidate effective for the treatment of neurodegenerative diseases such as Alzheimer's disease and amyotrophic lateral sclerosis (ALS) (PCT/KR2017/013444, PCT/KR2020/006680, U.S. Ser. No. 16/880,894). Specifically, trametinib induces neurogenesis by differentiating neural stem cells to neurons and protects neurons in the presence of neurotoxic materials by activating autophagic lysosome function. The effect of trametinib was also confirmed in 5XFAD, an Alzheimer's disease mouse, and SOD-G93A (B6SJL-Tg(SOD1-G93A)1Gur/J), an ALS model mouse.

### DISCLOSURE OF INVENTION

### Technical Problem

In applying MEK 1/2 inhibitors, including trametinib, as a treatment for neurodegenerative diseases, biomarkers that change in response to exposure to drugs in the body of an individual can provide useful information for managing the individual, such as determining the possibility of therapeutic effects, and deciding whether to continue drug treatment or adjust the dose. Therefore, the inventors sought to discover biomarkers that change sensitively in response to the administration of trametinib to 5XFAD, an Alzheimer's disease model mouse, and develop a predictive biomarker or pharmacodynamic or response biomarker that can be used in the treatment of neurodegenerative diseases with a drug containing a MEK 1/2 inhibitor as an active ingredient.

### Solution to Problem

The present invention is directed to a method of monitoring response to treatment with a MEK 1/2 inhibitor in a subject diagnosed with neurodegenerative disease, the method comprising measuring the level of at least one biomarker selected from the group consisting of osteopontin, synaptotagmin-1, apolipoprotein-E, cathepsin B, HLA-DOB (HLA class II histocompatibility antigen, DO beta chain), and neurofilament light chain in a biological sample of the subject obtained at one or more time points during or after the treatment. The level or the change thereof of said biomarkers provides information for one to evaluate response to the treatment with a MEK 1/2 inhibitor. The measured level of the biomarker measured can be compared with the level of the biomarker in a biological sample obtained from the subject before the treatment with the MEK 1/2 inhibitor and/or a biological sample from a healthy subject without the neurodegenerative disease. The change or difference in the level of the at least one biomarker in the biological sample obtained from the subject during or after the treatment with the MEK 1/2 inhibitor as compared to the level of the at least one biomarker in a biological sample obtained from the subject before the treatment with the MEK 1/2 inhibitor or a biological sample from a healthy subject without the neurodegenerative disease represents the response of the subject to the treatment with the MEK 1/2 inhibitor. The information provided by the method may be used to determine a subsequent dose or duration of treatment with the MEK 1/2 inhibitor. The information provided by the method may be used to determine whether to continue or stop the administration of the MEK 1/2 inhibitor.

In another aspect, the present invention is directed to a method of predicting response to treatment with a MEK 1/2 inhibitor in a subject with a neurodegenerative disease, comprising steps of measuring the level of at least one biomarker selected from the group consisting of osteopontin, synaptotagmin-1, apolipoprotein-E, cathepsin B, HLA-DOB, and neurofilament light chain in a biological sample of the subject obtained at one or more time points during or after the treatment, and evaluating the response of the subject to the treatment with the MEK 1/2 inhibitor based on the measured level of the biomarker. One can evaluate response to the treatment with the MEK 1/2 inhibitor from the level or the change thereof of said biomarkers. The method may further comprise determining a subsequent dose or duration of treatment with the MEK 1/2 inhibitor. The method may further comprise deciding whether to continue or stop the administration of the MEK 1/2 inhibitor.

The response evaluation comprises determining that the subject has responded to the treatment with the MEK 1/2 inhibitor if there is a change in the level of the at least one biomarker in a biological sample of the subject obtained during or after the treatment compared to the level of the at least one biomarker in a biological sample of the subject obtained before the treatment. The response evaluation may further comprise comparing the level of at least one biomarker in a biological sample of the subject obtained during or after the treatment with the MEK 1/2 inhibitor with the level in a biological sample of a healthy subject without the neurodegenerative disease. If the difference between the level of the biomarker during or after the treatment and that in a healthy subject is less than the difference between the level before the treatment and the level in the healthy subject, one can determine that the subject has shown a beneficial response to the treatment with the MEK 1/2 inhibitor.

In another aspect, the present invention is directed to a method of treating a neurodegenerative disease, comprising administration of a MEK 1/2 inhibitor at a daily effective dose that induces a change in the level of at least one biomarker selected from the group consisting of osteopontin, synaptotagmin-1, apolipoprotein-E, cathepsin B, HLA-DOB, and neurofilament light chain in a biological sample of a subject with the neurodegenerative disease.

In an aspect of the invention, the biological sample is obtained from a subject after administration of a MEK 1/2 inhibitor. In one aspect, the biological sample is obtained at multiple time points after administration of a MEK 1/2 inhibitor. In one aspect, the method of the present invention comprises measuring the level of the biomarker in a biological sample obtained from the subject before the administration of the MEK 1/2 inhibitor. In one aspect, the evaluation of response to a MEK 1/2 inhibitor comprises (a) measuring the level of at least one biomarker in a biological sample of a subject after administration of the MEK 1/2 inhibitor, and (b) comparing the level with that in a biological sample of the subject obtained before the administration, or (c) comparing the level with the level in a biological sample obtained from a healthy subject without the neurodegenerative disease.

In another aspect, the present invention is directed to a composition for use in evaluating response to treatment with a MEK 1/2 inhibitor in a subject with a neurodegenerative disease, comprising a probe specifically binding to at least one biomarker selected from the group consisting of osteopontin, synaptotagmin-1, apolipoprotein-E, cathepsin B, HLA-DOB, and neurofilament light chain. The probe may be an aptamer, peptide, antibody, or a fragment thereof that specifically binds to the biomarker.

### Advantageous Effects of Invention

The present invention provides a method of evaluating response to a MEK 1/2 inhibitor in a subject diagnosed with a neurodegenerative disease, thereby providing useful information for managing the individual, such as determining the possibility of therapeutic effects early on and deciding whether to continue the treatment or adjust the dose.

### BRIEF DESCRIPTION OF DRAWINGS

In the figures, “Veh” means a group treated with a vehicle, “Tra 0.05,” “Tra 0.1,” and “Tra 0.2” are respectively groups treated with 0.05, 0.1, and 0.2 mg/kg/day trametinib, “Don” means a group treated with donepezil, and “Don+Tra0.1” means a group treated with donepezil together with 0.1 mg/kg/day trametinib.

FIGS. 1A and 1B are graphs comparing the levels of Spp1 mRNA expression in the cerebral cortex of wild-type mice and 8-month-old 5XFAD mice (FIG. 1A) and the brain of 13-month-old 5XFAD mice (FIG. 1B) (n=2-3/group).

FIGS. 2A and 2B are graphs showing the change of osteopontin levels in the plasma of wild-type, 8-month-old 5XFAD mice (FIG. 2A), and 13-month-old 5XFAD mice (FIG. 2B) upon treatment of trametinib and/or donepezil. *P<0.05: 5XFAD-Veh vs. Tra 0.1, n=6-7/group.

FIG. 3 is a graph showing the change of synaptotagmin-1 levels in the plasma of wild-type and 8-month-old 5XFAD mice upon treatment of trametinib and/or donepezil. *P<0.05: 5XFAD-Veh vs. Don+Tra0.1, n=6-7/group.

FIG. 4 is a graph showing the change of apolipoprotein-E levels in the plasma of wild-type and 8-month-old 5XFAD mice upon treatment of trametinib and/or donepezil. **P<0.01: 5XFAD-Veh vs. Tra 0.2; *P<0.05: 5XFAD-Veh vs. Don+Tra0.1, n=6-7/group.

FIG. 5 is a graph showing the change of cathepsin B levels in the plasma of wild-type and 8-month-old 5XFAD mice upon treatment with trametinib or donepezil. *P<0.05: 5XFAD-Veh vs. Tra 0.1, 5XFAD-Veh vs. Don n=6-7/group.

FIG. 6 is a graph showing the change of cathepsin B levels in the plasma of 13-month-old 5XFAD mice upon treatment with trametinib.

FIGS. 7A and 7B are graphs showing the change of neurofilament light chain levels in the plasma of wild-type, 8-month-old 5XFAD mice (FIG. 7A), and 13-month-old 5XFAD mice (FIG. 7B) upon treatment of trametinib. *P<0.05: 5XFAD-Veh vs. Tra 0.1, n=6-7/group.

FIG. 8 is the immunofluorescence image showing the change in dendrite lengths in the cerebral cortex of wild-type and 8-month-old 5XFAD mice upon treatment of trametinib and/or donepezil. Antibody against Map2, a dendrite marker, was used for immunostaining.

FIG. 9 is a graph showing the dendrite lengths measured in FIG. 8. ***p<0.005: WT-Veh vs. 5XFAD-Veh, #p<0.005: 5XFAD-Veh vs. Don, ##p<0.005: 5XFAD-Veh vs. Tra 0.05, ###p<0.001: 5XFAD-Veh vs. Tra 0.1, Tra 0.2 or Don+Tra 0.1, n=3/group.

FIG. 10 indicates genes showing changes in the expression level in the cerebral cortex of 7-week-old normal mice after 1, 2, 3, and 4 weeks of administration of 0.1 mg/kg/day trametinib.

FIG. 11 is a graph comparing the levels of H2-Ob mRNA expression in the cerebral cortex of wild-type and 8-month-old 5XFAD mice upon treatment with trametinib.

### MODE FOR THE INVENTION

Unless defined otherwise, the technological and scientific terms used herein have a meaning that is commonly understood by those skilled in the art. Any methods and materials similar or equivalent to those described herein can be used for the implementation or experiment of the present invention.

### Definition

“MEK 1/2 inhibitor” is a compound that inhibits both MEK1 and MEK2, which are subtypes of MEK (mitogen-activated protein kinase kinase; also called MAP2K or MAPKK), a member of the MAP kinase (mitogen-activated protein kinase; MAPK) signal transduction pathway (also known as “MAPK/ERK pathway”) that follows in the sequence of Ras-Raf-MEK-ERK. MEK 1/2 inhibitor preferably has an IC50 value in the nM level and a difference of 10× or less in the IC50 values of MEK1 and MEK2, preferably 5× or less. For example, the MEK 1/2 inhibitor is trametinib, pimasertib (AS703026), AZD8330, binimetinib (MEK162, ARRY-162, ARRY-438162), refametinib (RDEA119, Bay 86-9766), PD318088, PD0325901, or R05126766.

Preferably, the MEK 1/2 inhibitor is trametinib (GSK 1120212, GSK1120212, JTP74057, or JTP-74057) represented by the following Formula 1. The chemical name is N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide. In the present invention, the compound of Formula 1 is used in the form of a free base or a pharmaceutically acceptable salt or solvate. Examples of possible solvates are hydrates, or other solvates such as a solvate of dimethyl sulfoxide, acetic acid, ethanol, nitromethane, chlorobenzene, 1-pentanol, isopropyl alcohol, ethylene glycol, 3-methyl-1-butanol, etc.

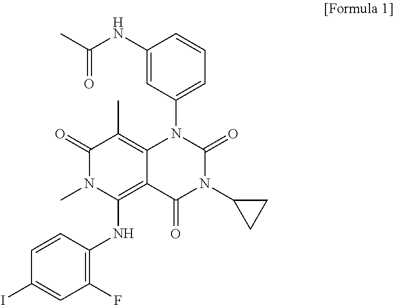 [SMILES: CC(=O)Nc1cccc(-n2c(=O)n(C3CC3)c(=O)c3c(Nc4ccc(I)cc4F)n(C)c(=O)c(C)c32)c1]

Osteopontin (OPN) is a protein encoded by the SPP1 gene and belongs to a family of secreted acidic proteins. It is an important factor in bone remodeling and is known to be widely expressed in immune cells such as macrophages, neutrophils, dendritic cells, microglia, and T/B cells. It functions as an immune modulator and has chemotactic properties. It was reported that OPN levels increase in the cerebrospinal fluid (CSF) and plasma or serum of Alzheimer's disease patients, especially with further disease progression (Sun et al., Elevated osteopontin levels in mild cognitive impairment and Alzheimer's disease. Mediators Inflamm. 2013: 615745 (2013)). The osteopontin level in healthy adults may vary between 31 and 200 ng/ml or higher depending on the measuring methods (Salem et al. Clinical Significance of Plasma Osteopontin Level as a Biomarker of Hepatocellular Carcinoma. Gastroenterology Res. 2013 October; 6(5): 191-199). A recent publication reports that the average level is 330 ng/ml (Nourkami-Tutdibi et al. Plasma levels of osteopontin from birth to adulthood. Pediatr Blood Cancer. 2020; 67: e28272).

Synaptotagmins are membrane trafficking proteins characterized by having an N-terminal transmembrane region, a variable linker, and two C-terminal C2 domains (C2A and C2B). There are 17 isoforms in the mammalian synaptotagmin family. Synaptotagmin-1 (Syt-1) is a protein on the presynaptic vesicle that plays an essential role in synaptic exocytosis. It has been reported to decrease in the brain cortical areas reflecting the progression of Alzheimer's disease and synapse damages (Blennow et al. (2018). Biomarkers for Alzheimer's disease: current status and prospects for the future. J Intern Med 284, 643-663). Since the detection of Syt1 in the CSF in the 1990s, high levels of Syt1 were reported in the CSF of Alzheimer's disease patients, suggesting the potential use of synaptotagmins as novel biomarkers for Alzheimer's disease (Ohrfelt et al. (2016). The presynaptic vesicle protein synaptotagmin is a novel biomarker for Alzheimer's disease. Alzheimers Res Ther 8, 41).

Apolipoprotein-E is a multifunctional protein involved in lipid metabolism, and its alteration is implicated in various neurodegenerative diseases. In particular, the c4 allele of Apolipoprotein E (ApoE4) is known as a major genetic risk factor for Alzheimer's disease. Due to the high correlation between the ApoE4 gene and Alzheimer's disease, many studies have been and are currently underway to measure the CSF and plasma ApoE levels for the development of biomarkers for Alzheimer's disease.

Cathepsin B is a lysosomal cysteine protease of 30 kDa that plays a role in degrading proteins that enter the lysosomal system from outside the cell via endocytosis or phagocytosis. It was reported to be elevated in the plasma and CSF of Alzheimer's disease patients (Morena et al. (2017). A comparison of lysosomal enzymes expression levels in peripheral blood of mild- and severe-Alzheimer's disease and MCI patients: Implications for regenerative medicine approaches. Int J Mol Sci 18(8): 1806; Sundelof et al. (2010). Higher cathepsin B levels in plasma in Alzheimer's disease compared to healthy controls. Journal of Alzheimer's Disease 22: 1223-1230; Zhang et al. Quantitative proteomics of cerebrospinal fluid from patients with Alzheimer's disease. J Alzheimers Dis. 2005; 7(2):125-33).

HLA-DOB is HLA (Human Leukocyte Antigen) HLA Class II histocompatibility antigen, DO beta chain coded by human HLA-DOB gene (mouse ortholog is H2-Ob). It belongs to the HLA class II beta chain paralogues. This class II molecule is a heterodimer consisting of an alpha (DOA) and a beta chain (DOB), both anchored in the membrane. It is located in intracellular vesicles. Class II molecules are expressed in antigen presenting cells (B lymphocytes, dendritic cells, macrophages). HLA-DOB is an important modulator in the HLA class II restricted antigen presentation pathway by interaction with the HLA-DM molecule in B-cells, and modifies peptide exchange activity of HLA-DM.

Neurofilaments are classed as type IV intermediate filaments found in the cytoplasm of neurons. They are protein polymers measuring 10 nm in diameter and many micrometers in length. Together with microtubules (˜25 nm) and microfilaments (7 nm), they form the neuronal cytoskeleton. Mammalian neurofilaments are heteropolymers of different proteins L (NfL), M (NfM), H (NfH), internexin-alpha, and peripherin. Among these, neurofilament light chain (NfL) is known as a promising fluid biomarker for following disease progression in Alzheimer's disease patients. It is reported that NfL is elevated in the CSF and plasma or serum of Alzheimer's disease patients (Lewczuk et al. (2018), Plasma neurofilament light as a potential biomarker of neurodegeneration in Alzheimer's disease. Alzheimers Res Ther 10(1):71). Axonal damage causes neurite components such as neurofilament light chain to be secreted into the extracellular space and as a result, NfL is secreted into body fluids such as CSF or plasma.

Neurodegenerative disease pertains to functional disorders in various systems such as motor control, cognition, perception, sensory function, and the autonomic nervous system due to the loss or decrease in neuronal function. Examples of neurodegenerative diseases include, but are not limited to, dementia, Alzheimer's disease, vascular dementia, frontotemporal dementia, Lewy body dementia, multiple system atrophy, corticobasal degeneration, progressive supranuclear palsy, Huntington's disease, amyotrophic lateral sclerosis (ALS, Lou-Gehrig's disease), primary lateral sclerosis, spinal muscular atrophy, progressive bulbar palsy (PBP), progressive muscular atrophy (PMA), pseudobulbar palsy, hereditary spastic paraplegia (HSP), cerebellar ataxia, Parkinson's disease, multiple sclerosis (MS), mild cognitive impairment (MCI), etc. Preferably, the neurodegenerative disease is Alzheimer's disease.

In one embodiment, neurodegenerative diseases involve abnormal activation of MAPK/ERK pathway. In one embodiment, neurodegenerative diseases involve abnormal autophagy-lysosomal function.

The term “treatment” or “treatment reaction” refers to all activities that change a subject suspected or diagnosed with a neurodegenerative disease in a beneficial way, such as improving symptoms, delaying progression of the disease, recovering from neuronal injury in the subject by administration of a MEK 1/2 inhibitor.

The term “subject” or “individual” is not particularly limited and may be any subject that needs treatment with a MEK 1/2 inhibitor.

The term “probe” refers to a material that specifically binds to a target molecule to be detected in a sample, and is a broad concept including a probe that specifically attaches to a target molecule through the binding and identifies and/or detects the target molecule. The type of probe is not particularly limited and may be any substance commonly used in the art.

### Dose

MEK 1/2 inhibitors such as trametinib are administered in a therapeutic effective dose. In the present invention, the therapeutic effective dose or amount is a dose effective for treating or alleviating the neurodegenerative disease of a subject or delaying the progression of the disease. In one embodiment, the therapeutic effective dose is a dose effective to treat or alleviate Alzheimer's disease or delay the progression of the disease.

In one embodiment, the therapeutic effective dose is a dose sufficient to induce neural differentiation. In one embodiment, the therapeutic effective dose is a dose sufficient to induce neural regeneration. In one embodiment, the therapeutic effective dose is a dose sufficient to induce autophagy-lysosome activity. In one embodiment, the therapeutic effective dose is a dose sufficient to enhance autophagosome-lysosome fusion.

In one embodiment, the therapeutic effective dose is a dose sufficient to induce a change in the level of at least one biomarker. In one embodiment, the therapeutic effective dose is a dose sufficient to induce a change in the level of at least one biomarker in a biological sample obtained from an individual. In one embodiment, the biomarker is selected from the group consisting of osteopontin, synaptotagmin-1, apolipoprotein-E, cathepsin-B, HLA-DOB, and neurofilament light chain. The biomarkers are used to evaluate response to MEK 1/2 inhibitors such as trametinib.

In one embodiment, the MEK 1/2 inhibitor is administered at a dose that increases or decreases the level of at least one biomarker in an individual's biological sample by at least 0.5%, at least 1%, at least 1.5%, at least 2%, at least 2.5%, at least 5%, at least 10%, at least 20%, at least 30%, at least 40%, at least 50%, at least 60%, at least 70%, at least 80%, at least 90%, at least 95%, or at least 99% as compared to the level before or without the administration of the MEK 1/2 inhibitor. In one embodiment, the MEK 1/2 inhibitor is administered at a dose maintaining the level of at least one biomarker at the level of a healthy individual without the disease.

In one embodiment, trametinib is administered at a dose of 0.1 to 2 mg/day. In one embodiment, trametinib is administered at a dose of 0.2 to 1.5 mg/day. In one embodiment, trametinib is administered at a dose of 0.25 to 1 mg/day. In one embodiment, trametinib is administered at a dose of 0.25 to 0.5 mg/day. In one embodiment, trametinib is administered at a dose of 0.5 to 1 mg/day. In one embodiment, trametinib is administered at a dose of 1 to 2 mg/day. In one embodiment, trametinib is administered at a dose of 0.1 mg/day, 0.125 mg/day, 0.2 mg/day, 0.25 mg/day, 0.3 mg/day, 0.4 mg/day, 0.5 mg/day, 0.6 mg/day, 0.7 mg/day, 0.75 mg/day, 0.8 mg/day, 0.9 mg/day, 1 mg/day, 1.5 mg/day, or 2 mg/day.

### Duration

In one embodiment, MEK 1/2 inhibitors are administered for a period sufficient to induce neural differentiation. In one embodiment, MEK 1/2 inhibitors are administered for a period sufficient to induce neural regeneration. In one embodiment, MEK 1/2 inhibitors are administered for a period sufficient to induce autophagy-lysosome activity. In one embodiment, MEK 1/2 inhibitors are administered for a period sufficient to enhance autophagosome-lysosome fusion.

In one embodiment, MEK 1/2 inhibitors are administered for a period sufficient to induce a change in the level of at least one biomarker in a biological sample obtained from an individual. In one embodiment, the biomarker is selected from the group consisting of osteopontin, synaptotagmin-1, apolipoprotein-E, cathepsin B, HLA-DOB, and neurofilament light chain. In one embodiment, the biomarker may be used to evaluate a response to a MEK 1/2 inhibitor such as trametinib.

In one embodiment, MEK 1/2 inhibitors are administered for a period sufficient to increase or decrease the level of at least one biomarker by at least 0.5%, at least 1%, at least 1.5%, at least 2%, at least 2.5%, at least 5%, at least 10%, at least 20%, at least 30%, at least 40%, at least 50%, at least 60%, at least 70%, at least 80%, at least 90%, at least 95%, or at least 99% as compared to the level before or without the administration of the MEK 1/2 inhibitors. In one embodiment, MEK 1/2 inhibitors are administered for a period sufficient to recover the level of at least one biomarker to the level of a healthy individual without the disease.

In one embodiment, individuals are administered a therapeutic effective dose of trametinib for at least 4 weeks. In one embodiment, trametinib is administered for at least 5 weeks, at least 6 weeks, at least 7 weeks, at least 8 weeks, at least 9 weeks, and at least 10 weeks. In one embodiment, trametinib is administered for at least 1 month, at least 2 months, at least 3 months, at least 4 months, at least 5 months, at least 6 months, and at least 12 months.

### Detection of Biomarkers

One aspect of the present invention relates to a method for evaluating response to treatment with MEK 1/2 inhibitors such as trametinib. The method includes measuring the level of at least one biomarker selected from the group consisting of osteopontin, synaptotagmin-1, apolipoprotein-E, cathepsin B, HLA-DOB, and neurofilament light chain in a biological sample obtained from an individual with a neurodegenerative disease. The level of the above biomarker or a change thereof may provide information for evaluating response to treatment with a MEK 1/2 inhibitor. The information provided by the method may be used to determine a subsequent dose or duration of treatment with the MEK 1/2 inhibitor. The information provided by the method may be used to determine whether to continue or stop the treatment with the MEK 1/2 inhibitor.

In one embodiment, the present invention comprises a step of measuring the level of at least one biomarker in a biological sample obtained from an individual. The level of the biomarker can be measured using various protein analysis methods known in the art. For example, it can be measured by contacting the sample with an antibody that specifically binds to the biomarker under a condition sufficient to form an antibody-marker complex and detecting the complex. The presence of biomarkers can be detected in many ways, including western blotting, enzyme-linked immunosorbent assay (ELISA), immunoelectrophoresis, protein immunoprecipitation, protein immunostaining, two-dimensional SDS-PAGE, fluorescence-activated cell sorting (FACS), flowcytometry, confocal imaging, and mass spectroscopy.

In one embodiment, the level of biomarkers in the sample can be measured by a solid-phase sandwich ELISA. For example, a first capture antibody that specifically and sensitively binds to a target biomarker protein is coated onto the well of a microplate. A sample is added to the well so that the target biomarker in the sample can be attached to the capture antibody. By adding a second detection antibody to the well, the target biomarker binds to the second antibody to form a sandwich structure between the first capture and second detection antibodies. The target biomarker can be detected by an enzyme conjugated to the second detection antibody through an enzyme-substrate reaction.

In one embodiment, if the ELISA method is not sensitive enough, another method can be used. For example, neurofilament light chains can be detected using Simoa® (Single molecular array; Quanterix, Lexington, Mass., USA) technology. In addition, when high sensitivity, specificity, and consistency are needed, such as in the analysis of cerebrospinal fluid, an electro-chemiluminescence immunoassay (ECLIA) can be used. For example, synaptotagmin-1 in cerebrospinal fluid can be measured with a method described in Ohrfelt et al., The pre-synaptic vesicle protein synaptotagmin is a novel biomarker for Alzheimer's disease. Alzheimer's Research & Therapy (2016) 8:41.

In one embodiment, the level of each biomarker can be measured using a commercially available ELISA kit. In one embodiment, osteopontin concentration in a biological sample can be measured according to the manufacturer's instructions using a commercially available Human Osteopontin ELISA kit (Immuno-Biological Laboratories Co. Ltd, Gumma, Japan, or Assay Designs, Inc. Ann Arbor, Mich., USA, etc.). For example, serum and cerebrospinal fluid samples are diluted at a ratio of 1:20 and 1:50, respectively, with an assay buffer contained in the kit, and incubated at 37° C. for 1 hour in a microtiter plate pre-coated with a polyclonal N-terminal capture anti-OPN antibody (Assay Designs). The plate is then washed and incubated at 4° C. for 30 minutes with an OPN-specific monoclonal antibody labeled with horseradish peroxidase (Assay Designs). After washing, the well is incubated with the tetramethylbenzidine-H₂O₂ solution for 30 minutes. A solution containing IN sulfuric acid is added to stop the color development reaction. Optical density at 450 nm is measured. The OPN concentration is calculated using the standard curve of the human recombinant OPN provided by the manufacturer.

In one embodiment, the level of the biomarker may be measured by mass spectrometry. The method for quantifying proteins or peptides using mass spectrometry is, for example, TMT (tandem mass tags), iTRAQ (Isobaric Tags for Relative and Absolute Quantification), MRM (Multiple Reaction Monitoring), AQUA (Absolute QUAntification of proteins), or SWATH (Sequential Window Acquisition of all THeoretical Mass Spectra). The absolute concentration of a particular target protein can be measured by spiking-in a known concentration of a reference peptide or protein labeled with a heavy isotope. The usefulness of this concept was demonstrated in a method using an appropriate reference material selected from a large library of protein fragments as described in Edfors et al. Screening a Resource of Recombinant Protein Fragments for Targeted Proteomics. J. Proteome Res. 2019, 18, 2706-2718, or in a method using SISCAPA (stable isotope standards and capture by anti-peptide antibodies) (Anderson et al. Multiplexed measurement of protein biomarkers in high-frequency longitudinal dried blood spot (DBS) samples: characterization of inflammatory responses. bioRxiv 2019, DOI: 10.1101/643239).

The level of the biomarker may be measured at various time points, and the amounts measured at different time points may be compared with each other. The changes in the biomarker level over time can be used to determine, monitor, or predict the therapeutic effectiveness and/or therapeutic reactivity of MEK 1/2 inhibitors in individuals.

In one embodiment, the level of the biomarker is measured in a sample obtained after administration of a MEK 1/2 inhibitor such as trametinib. Samples can be obtained at one or more time points after starting administration of the MEK 1/2 inhibitor. For example, samples can be obtained at one or more time points after 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks after starting administration of the MEK 1/2 inhibitor. In one embodiment, samples can be obtained at one or more multiple time points after 1 month, 2 months, 3 months, 4 months, 5 months, 6 months, 9 months, or 12 months after starting the administration of a MEK 1/2 inhibitor.

In one embodiment, the level of biomarkers is measured in a control sample obtained before starting the administration of a MEK 1/2 inhibitor. In one embodiment, the level of biomarkers is measured in a sample obtained from a healthy individual without the disease. In one embodiment, the level of biomarkers measured in a sample obtained after administration of the MEK 1/2 inhibitor is compared with the level in a control sample obtained before the start of administration (or a sample obtained from healthy individuals). Information on the differences and/or changes obtained from the comparison of the biomarker levels can be used to evaluate a response to MEK 1/2 inhibitor treatment. In one embodiment, the level of the biomarkers can be used to determine the appropriate dose and duration of treatment with a MEK 1/2 inhibitor to achieve the desired effect of treating or delaying the disease. In one embodiment, the levels of the biomarker are analyzed over time. In one embodiment, the level of the biomarkers may be used to determine a method of subsequent administration of a MEK 1/2 inhibitor, for example, to determine the duration of administration and dose of a MEK 1/2 inhibitor. In one embodiment, the level of the biomarkers may be used to select subjects that are likely to obtain beneficial effects after the administration of a MEK 1/2 inhibitor.

Biological samples for testing biomarkers can be obtained by a well-known method in the art. For example, a biological sample includes an individual's body fluids or secretions, such as blood, cerebrospinal fluid, urine, body secretions, saliva, feces, pleural fluid, lymphatic fluid, sputum, ascites, prostate fluid, or other secretions or derivatives thereof. Blood is selected from whole blood, plasma, serum, peripheral blood mononuclear cells (PBMC), or any component of blood.

Another aspect of the present invention provides a composition or kit for evaluating response to treatment with a MEK 1/2 inhibitor such as trametinib, comprising as an active ingredient, a probe capable of specifically detecting a biomarker. In one embodiment, the probe capable of detecting a biomarker protein may be an aptamer, peptide, antibody, or fragment thereof that specifically binds to the protein. They may be manufactured according to conventional methods in the art. The form of the antibody includes a polyclonal antibody or a monoclonal antibody, and includes all immunoglobulin antibodies. The fragment of the antibody may be any one selected from the group consisting of scFv, Fab, Fab′, and F(ab)′.

The kit may include a carrier partitioned to accommodate one or more containers, such as a vial, a tube, etc., and each container includes one of the separate components used in this invention. For example, one of the containers includes a probe that is labeled or can be detectably labeled. The probe may be an antibody, peptide, or polynucleotide specific to a protein or mRNA. The kit typically contains the above containers and includes one or more other containers comprising things that are needed in commercial and user perspectives, such as buffers, diluents, filters, needles, syringes, and packaging inserts with instructions for use. The container may be labeled to indicate a special use of the composition or instructions for in vivo or in vitro applications.

For example, the kit includes a container, a label on the kit, and a composition in the container; the composition includes a first antibody that binds to a protein biomarker; the label on the container indicates that the composition can be used to detect a target protein in a sample; and the kit includes instructions for using the antibody to detect the target protein in a specific type of samples. The kit may further include materials and instructions necessary to prepare a sample and apply the sample to the antibody. The kit may include both primary and secondary antibodies, and the secondary antibody is conjugated to a color development label or an enzyme.

Hereinafter, the present invention will be described in detail with examples. However, the examples are for illustrative purposes only, and do not limit the scope of the invention.

### EXAMPLES

### Example 1

### Animals and Drug Treatments

5-month-old 5XFAD B6SJL-Tg mice (APPSwF1Lon,PSEN1*M146L*L286V) were orally administered vehicle or 0.05 mg/kg/day, 0.1 mg/kg/day or 0.2 mg/kg/day of trametinib (micronized) for 2.5 months daily (8-months-old upon completion of treatment, hereinafter referred to as “8-month-old 5XFAD mouse”). 2 mg/kg/day of donepezil was administered intraperitoneally for 2.5 months daily. Age-matched wild-type (WT) mice were orally administered vehicle for 2.5 months. Blood was obtained from all the mice at the age of 8 months after completion of the treatment and collected in EDTA tubes, which were centrifuged to obtain plasma. Mice were sacrificed by the perfusion method, and the brain cortex was extracted and immediately frozen.

12-month-old 5XFAD mice were orally administered vehicle or 0.1 mg/kg/day of trametinib (corn oil based) for 1 month daily (13-months-old upon completion of treatment, hereinafter referred to as “13-month-old 5XFAD mouse”). Blood was obtained from the 13-month-old mice after completion of the treatment and collected in EDTA tubes, which were centrifuged to obtain plasma. Mice were sacrificed, and the brain cortex was extracted, washed with PBS, and immediately frozen.

For whole cell RNA sequencing study, 6-month-old C57BL/6 mice (n=3 per group) were orally administered 0.1 mg/kg/day of trametinib (4% DMSO+96% corn oil) daily. Mice were sacrificed after 1, 2, 3, or 4 weeks of administration. Whole brain was extracted.

### Example 2

### Single Cell RNA Sequencing

Single-cell RNA sequencing analysis was performed on the 8-month-old 5XFAD mouse and wild type mouse brain cortex samples. The cortex was microdissected in HABG (Hibernate A buffer including B27 supplement and glutamine) media and digested in papain solution for 20 min at 37° C. (Brewer et al (2007). Isolation and culture of adult neurons and neurospheres. Nat Protoc 2, 1490-1498). Using a Pasteur pipette, the digested cortex was gently triturated. Cells were isolated by the density gradient method using OptiPrep (Sigma-Aldrich, D1556).

The cell suspension was diluted with Drop-seq buffer, and Drop-seq was performed using the Chromium™ Single Cell 3′ v2 Reagent Kit (Campbell et al. (2017). A molecular census of arcuate hypothalamus and median eminence cell types. Nat Neurosci 20, 484-496). Libraries were sequenced on the Illumina HiSeq X Ten, and Read 1 was 16 bp (10×™ Barcode and 10 bp UMI). Cells with either very low or too high mRNA content (to only include: 200<gene number <3,000) or a high fraction of mitochondrial encoded transcripts (>20%) were filtered out. Using 20,056 cells, raw sequence data were aligned to the mouse (mm10) genome, and analyses including PCA, t-SNE, and graph-based clustering were performed with the Cell Ranger Single-Cell software.

Microglial population was markedly increased in the vehicle treated 8-month-old 5XFAD mouse cortex samples compared to the wild type (WT)-vehicle treated group. The microglial population was then seen to decrease in the group treated with 0.1 mg/kg/day of trametinib (5XFAD-Tra 0.1 group) when compared to the 5XFAD-vehicle group (Table 1).

In the microglia cluster, genes that are expressed differentially between the experimental groups were identified. Cst7, Spp1, Apoe, Lpl, Fabp5, Mif, Syngr1, and Cts1 underwent at least 2-fold increase, while Malat1 and Cebpb showed at least 1.5-fold decrease in the 5XFAD-vehicle group compared to the WT-vehicle group. Ptgds, Rpl10-ps3, Rpl9-ps6, Acta2 underwent at least 1.5-fold increase in the 5XFAD-Tra 0.1 group compared to the 5XFAD-vehicle group, while Wfdc17 and Spp1 showed at least 1.5-fold decrease.

Notably, expression of the Spp1 gene increased in the 5XFAD-vehicle group compared to the WT group and decreased in the 5XFAD-Tra 0.1 group.

TABLE 1
Number of single cells analyzed

| Type of cells | 5XFAD-Tra 0.1 | 5XFAD-vehicle | WT-vehicle |
|---|---|---|---|
| Astrocyte | 48 | 3 | 10 |
| Endothelial | 1150 | 892 | 263 |
| M. Neuron | 43 | 53 | 34 |
| (mature neuron) | | | |
| M1 (microglia) | 2952 | 9324 | 57 |
| ODC | 457 | 249 | 269 |
| (oligodendrocyte) | | | |
| Pericyte | 141 | 186 | 30 |
| SMC (smooth | 1588 | 1310 | 997 |
| muscle cell) | | | |

TABLE 2
Genes differentially expressed between the experimental
groups in the microglia cluster

| 5XFAD-vehicle vs. WT-vehicle | | 5XFAD-Tra 0.1 vs. 5XFAD-vehicle | |
|---|---|---|---|
| Gene | Fold change | Gene | Fold change |
| Cst7 | 12.1871 | Ptgds | 1.70411 |
| Spp1 | 5.00804 | Rpl10-ps3 | 1.63168 |
| Apoe | 3.50348 | Rpl9-ps6 | 1.59081 |
| Lpl | 3.16689 | Acta2 | 1.513 |
| Fabp5 | 2.98236 | Cst7 | −1.1315 |
| Mif | 2.90452 | Wfdc17 | −1.5194 |
| Syngr1 | 2.77546 | Spp1 | −1.5648 |
| Ctsl | 2.75453 | | |
| Malat1 | −1.97444 | | |
| Cebpb | −2.5319 | | |

In addition to the microglial cells, the other brain cell types were also analyzed for the genes differentially expressed between the experimental groups. Genes were selected based on the criteria: genes showing increase or decrease of the expression level in the 5XFAD-vehicle group compared to wild type (Fold change 5XFAD-vehicle vs. WT>1.3) and showing recovery of the expression level in the 5XFAD mice group treated with 0.1 mg/kg/day of trametinib to the level of the corresponding brain cell of the WT-vehicle group (Fold change 5XFAD-Tra 0.1 vs. WT 1).

| | | Log₂(5XFAD- | | |
|---|---|---|---|---|
| | Log₂(5XFAD- | Tra0.1/5XFAD- | | |
| Gene | Veh/WT) | Veh) | Cell | Protein |
| Syt1 | −1.60980888 | 1.127259139 | M. Neu | Synaptotagmin-1 |
| Dbi | 3.110758452 | −1.719467503 | M. Neu | Acyl-CoA- |
| | | | | binding |
| | | | | protein |
| Spp1 | 2.324246313 | −0.645960417 | M | Osteopontin |
| | | | | (OPN) |
| CTSE | 0.949084768 | −0.542999502 | M | Cathepsin E |
| CTSB | 1.148084892 | 0.818183775 | Endo | Cathepsin B |
| ApoE | 1.17527371 | −0.517716873 | Astro | Apolipoprotein E |

M. Neu: mature neuron;
M: microglia,
Astro: Astrocyte,
Endo: Endothelial

### Example 3

### Change in Biomarkers in the Brain Tissue and Plasma of 5XFAD Mice

ELISA was performed to detect changes in the level of biomarkers in the plasma of 5XFAD mice. The following ELISA kits were used for each of the biomarkers:
Neurofilament-light chain (NfL): Cloud-Clone, SEE038Mu
Cathepsin B (CTSB): Novus, NBP2-67254
Osteopontin (OPN): LS bio, LS-F26046-1
Synaptotagmin-1 (Syt1): LS bio, LS-F30963-1
Apolipoprotein-E (ApoE): Novus, NBP2-66739

NfL, CTSB and ApoE were detected by colorimetric methods. OPN and Syt1 were detected by chemiluminescent methods. ELISA was performed according to the ELISA kit manufacturer's instructions.

In addition, qRT-PCR was performed to measure the mRNA expression level in the brain tissue of each animal. Total RNA was extracted from each sample using TRIzol (Invitrogen, 15596026). Reverse transcription was performed using M-MLV reverse transcriptase (Invitrogen, 28025013). qRT-PCR was performed using the SYBRTM Green PCR master mix (Thermo, 4367659) according to the manufacturer's instructions. Results were expressed relative to the housekeeping gene GAPDH (Glyceraldehyde-3-Phosphate Dehydrogenase).

For immunohistochemical analysis, the brain tissue was fixed with 4% formaldehyde (PFA), embedded in paraffin, and sagittally cut into 5 μm sections. Sections were deparaffinized, and antigen retrieval was performed in 10 mM sodium citrate buffer (Tri-sodium citrate, pH 6.0) (Sigma-Aldrich, S4641). For immunostaining, the sections were incubated with anti-Map2 antibody (Millipore, Mab3418). Afterward, the sections were incubated with Alexa Fluor 488-conjugated anti-mouse (Thermo, a21121), IgG secondary antibody. The sections were counterstained with DAPI. The immunofluorescent images were captured using a LSM700 Laser-Scanning confocal microscope (Carl Zeiss).

### Osteopontin

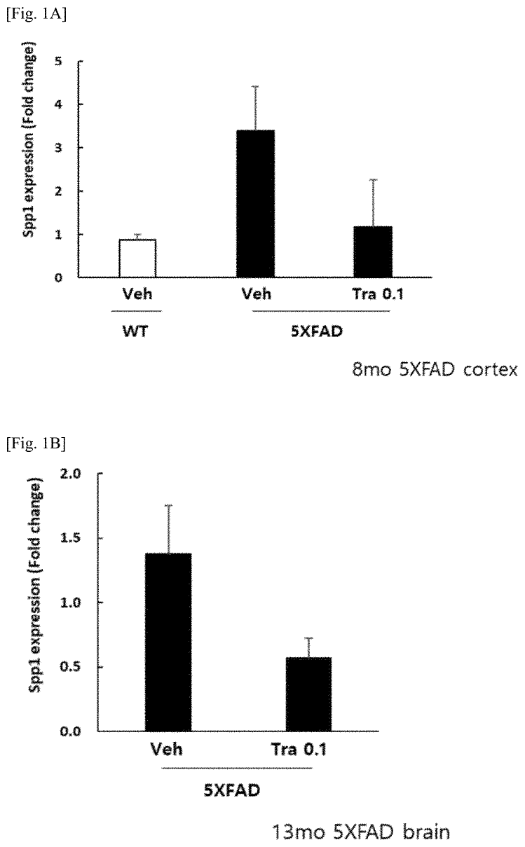

Expression level of Spp1 was analyzed in the 8-month-old 5XFAD mice cortex. Spp1 level showed an increasing trend in the 5XFAD-vehicle group compared with the WT-vehicle group and then a decreasing trend in the 5XFAD-Tra 0.1 group compared with the 5XFAD-vehicle group (FIG. 1A). Spp1 level in the 13-month-old 5XFAD mice brain showed a decreasing trend in the 5XFAD-Tra 0.1 group compared with the 5XFAD-vehicle group (FIG. 1B).

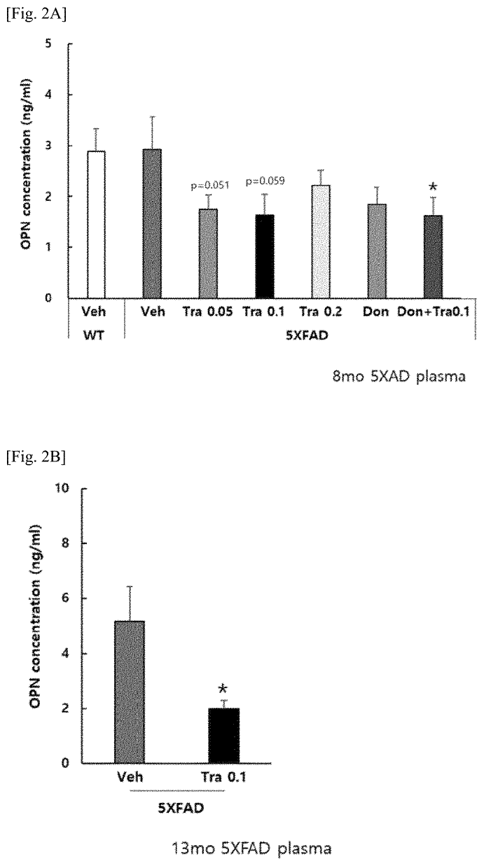

Plasma osteopontin (OPN) levels showed a decreasing trend in 8-month-old 5XFAD groups administered with 0.05 and 0.1 mg/kg/day trametinib in comparison with the 5XFAD-vehicle group (FIG. 2A, decreased by 40% and 43.9%, respectively). The OPN level decreased by 44.6% with statistical significance in the group administered with both donepezil and trametinib 0.1 mg/kg/day in comparison with the 5XFAD-vehicle group (FIG. 2A). In the 13-month-old 5XFAD mice, plasma OPN level decreased by 61.5% with statistical significance in the trametinib 0.1 mg/kg/day-administered group when compared to the vehicle group (FIG. 2B).

### Synaptotagmin-1

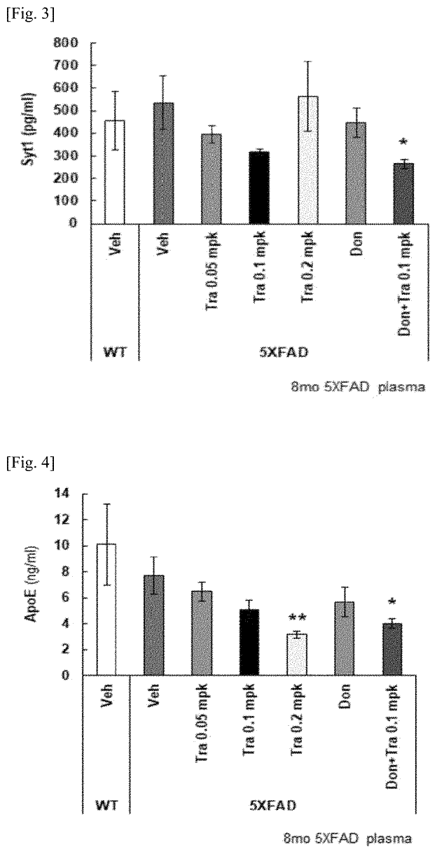

Plasma synaptotagmin-1 (Syt1) levels showed a decreasing trend in the 8-month-old 5XFAD groups administered with 0.05 and 0.1 mg/kg/day trametinib in comparison with the 5XFAD-vehicle group (decreased by 26.2% and 41.0%, respectively). Syt1 level decreased by 50.7% with statistical significance in the group administered with both donepezil and trametinib 0.1 mg/kg/day in comparison with the 5XFAD-vehicle group (FIG. 3).

### Apolipoprotein-E

Plasma apolipoprotein-E (APOE) level showed a decreasing trend in 8-month-old 5XFAD mice administered with 0.1 mg/kg/day trametinib (decreased by 34.3%) in comparison with the 5XFAD-vehicle group, with the decrease reaching statistical significance in the trametinib 0.2 mg/kg-administered group (a decrease of 58.9%). The group administered with both donepezil and 0.1 mg/kg/day trametinib also exhibited a statistically significant decrease of 47.3% in APOE level in comparison to the 5XFAD-vehicle group (FIG. 4).

### Cathepsin B

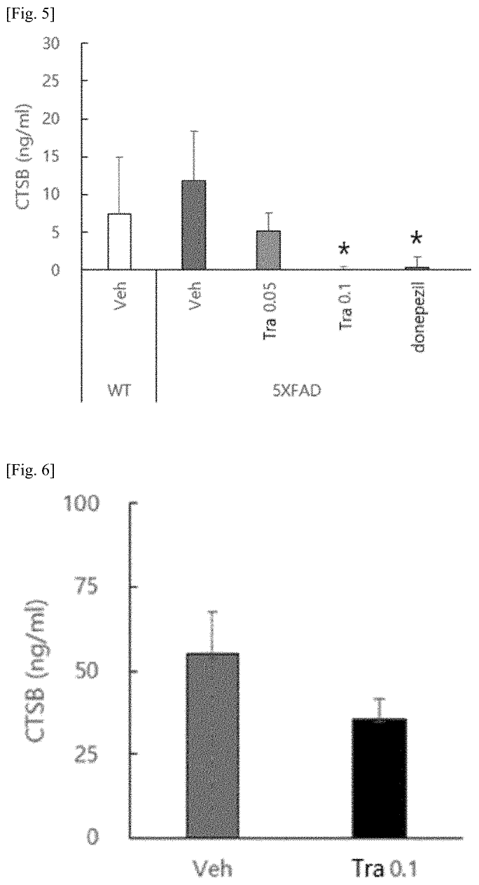

Plasma cathepsin B levels showed a decreasing trend in 8-month-old 5XFAD groups administered with 0.05 and 0.1 mg/kg/day trametinib in comparison with the 5XFAD-vehicle group (decreased by 62.4% and 99%, respectively), with the decrease reaching statistical significance in the trametinib 0.1 mg/kg-administered group. The donepezil-administered group also exhibited a statistically significant decrease in cathepsin B level in comparison to the 5XFAD-vehicle group (a decrease of 97.6%) (FIG. 5). In the 13-month-old 5XFAD mice, plasma cathepsin B level showed a decreasing trend compared to the 5XFAD-vehicle group (decreased by 35%, FIG. 6).

### Neurofilament Light Chain

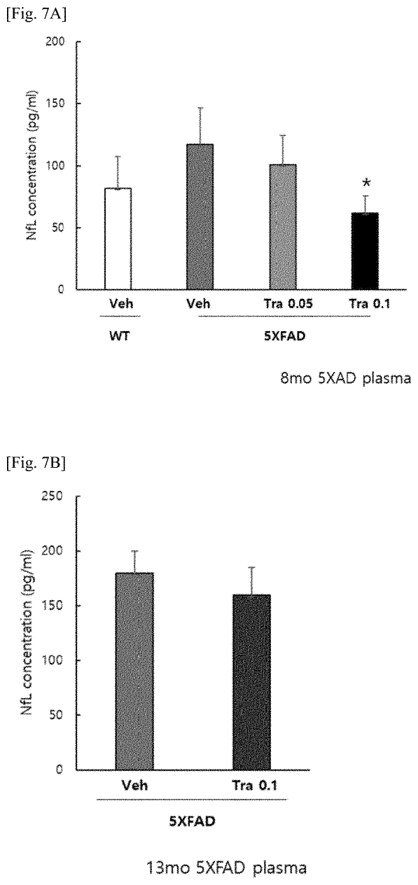

When plasma neurofilament light chain (NfL) levels were measured in the 8-month-old 5XFAD mice, NfL levels showed a statistically significant decrease of 47.4% in mice administered with 0.1 mg/kg/day trametinib in comparison to the 5XFAD-vehicle group (FIG. 7A). Plasma NfL levels measured in 13-month-old 5XFAD mice did not show a significant difference between the vehicle and trametinib-administered groups (FIG. 7B).

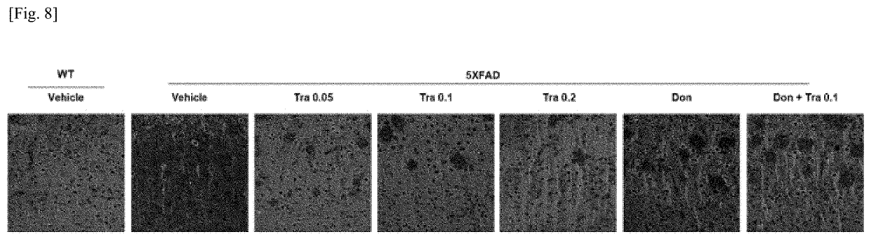

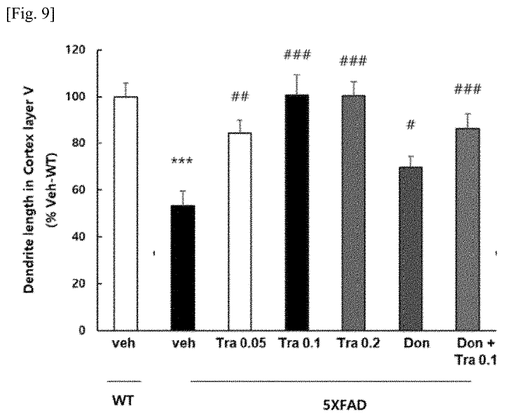

The decrease in the plasma NfL level in the 8-month-old trametinib-administered 5XFAD mice appears to relate to the protection of neurites of neurons in the brain by trametinib. Our immunohistochemical studies showed that the neurite marker (Map2) was increased in the brain tissue of trametinib-administered 8-month-old 5XFAD mice (FIGS. 8 and 9).

### HLA-DOB

C57BL/6 mice were administered with 0.1 mg/kg/day of trametinib and sacrificed after 1, 2, 3, or 4 weeks of administration. Whole cell RNA sequencing analysis was performed with the RNA isolated from the whole brain of the mice to observe the change of genes in the brain during the administration period.

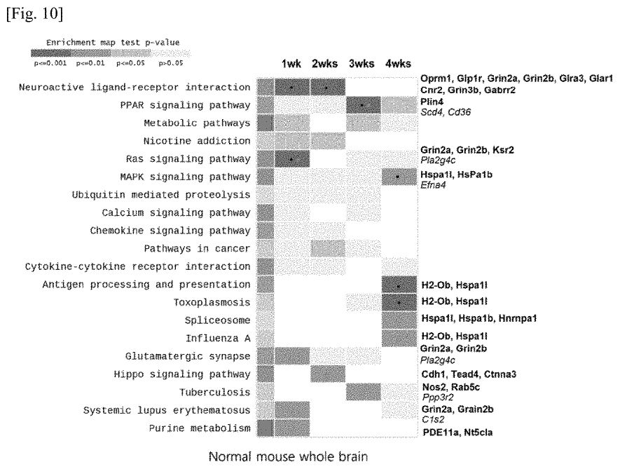

The whole cell RNA sequencing was performed as follows. RNA was isolated from the whole brains of the mice, and cDNA libraries for RNA sequencing were prepared using the TruSeq Stranded mRNA Prep Kit (Illumina, San Diego, Calif.) according to the manufacturer's guidelines. The libraries were sequenced on the Illumina Nextseq500 platform, and the reads were mapped to the reference Mouse mm10 genome using Tophat v2.0.13. After read mapping, transcript assembly was performed using StringTie program. The expression profile for each sample was obtained, and RPKM (Read per Kilobase per million mapped reads) values were calculated based on the transcripts/genes. To compare the gene expression levels in each experimental group, DEG (Differentially Expressed Genes) were analyzed using DESeq2. As a result, 196 genes satisfying |fc|>=2, p value <0.05 in at least one comparison set were extracted. The extracted gene information was checked in KEGG database (http://www.kegg.jp/kegg/pathway.html) to identify which pathway the genes belong to. Genes showing at least a twofold increase in the expression level in the corresponding pathway were marked bold, and those showing at least a twofold decrease were marked italic on the right side of the heatmap (FIG. 10). Notably, the H2-Ob level remarkably increased in 3 and 4 weeks after the administration.

TABLE 4

| | |
|---|---|
| Genes showing an increase in | Oprm1, Glp1r, Grin2a, Grin2b, Glra3, |
| the expression level in weeks | Glra1, Cnr2, Grin3b, Gabrr2, Ksr2, Cdh1, |
| 1 and 2 | Tead4, Ctnna3, PDE11a, Nt5cla |
| Genes showing a decrease in | Pla2g4c, C1s2 |
| the expression level in weeks | |
| 1 and 2 | |
| Genes showing an increase in | Plin4, H2-Ob, Hspa1l, HSPa1b, Hnmpa1, |
| the expression level in weeks | Nos2, Rab5c |
| 3 and 4 | |
| Genes showing a decrease in | Efna4, Scd4, Cd36, Ppp3r2 |
| the expression level in weeks | |
| 3 and 4 | |

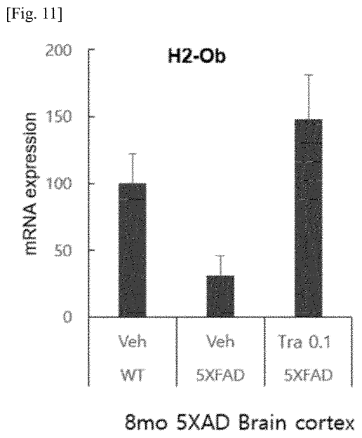

When the mRNA expression levels of H2-Ob were compared in the brain cortex of 8-month-old 5XFAD mice, the level tended to decrease in the 5XFAD-vehicle group compared to the wild type-vehicle group. In contrast, the level significantly increased in the 5XFAD administered with 0.1 mg/kg/day trametinib compared to the 5XFAD-vehicle group (FIG. 11). H2-Ob in mice corresponds to HLA-DOB in humans.

1. A method of detecting a biomarker in a subject with a neurodegenerative disease, comprising detecting at least one biomarker selected from the group consisting of osteopontin, synaptotagmin-1, apolipoprotein-E, cathepsin B, HLA class II histocompatibility antigen, DO beta chain (HLA-DOB), and neurofilament light chain in a biological sample obtained from the subject during or after treatment of the subject with a compound inhibiting both mitogen activated protein kinase (MEK) 1 and 2 (MEK 1/2 inhibitor).

2. The method according to claim 1, wherein the method comprises detecting osteopontin, synaptotagmin-1, apolipoprotein-E, cathepsin B, HLA-DOB, and neurofilament light chain in the biological sample.

3. The method according to claim 1, wherein the MEK 1/2 inhibitor is trametinib.

4. The method according to claim 1, wherein the biological sample is obtained from the subject at different time points.

5. The method according to claim 1, wherein the detecting is performed using a probe comprising an aptamer, peptide, antibody, or a fragment thereof that specifically binds to the at least one biomarker.

6. The method according to claim 1, wherein the biological sample is blood or cerebrospinal fluid.

7. The method according to claim 6, wherein the blood is selected from the group consisting of whole blood, plasma, serum, and peripheral blood mononuclear cells (PBMC).

8. The method according to claim 1, wherein the neurodegenerative disease is selected from the group consisting of dementia, Alzheimer's disease, vascular dementia, frontotemporal dementia, Lewy body dementia, multiple system atrophy, corticobasal degeneration, progressive supranuclear palsy, Huntington's disease, amyotrophic lateral sclerosis (ALS, Lou-Gehrig's disease), primary lateral sclerosis, spinal muscular atrophy, progressive bulbar palsy (PBP), progressive muscular atrophy (PMA), pseudobulbar palsy, hereditary spastic paraplegia (HSP), cerebellar ataxia, Parkinson's disease, multiple sclerosis (MS), and mild cognitive impairment (MCI).

9. The method according to claim 1, wherein the detecting comprises measuring a level of the at least one biomarker.

10. The method according to claim 9, comprising comparing the measured level of the at least one biomarker with a control level of the at least one biomarker in a biological sample obtained from the subject before the treatment with the MEK 1/2 inhibitor or in a biological sample from a healthy subject without the neurodegenerative disease.

11. The method according to claim 10, wherein the change or difference in the level of the at least one biomarker in a biological sample obtained from the subject during or after the treatment with the MEK 1/2 inhibitor compared to the control level represents a response of the subject to the treatment with a MEK 1/2 inhibitor.

12. The method according to claim 11, wherein the reduction in the difference between the level of the at least one biomarker detected during or after the treatment with the MEK 1/2 inhibitor and the level in the healthy subject as compared to the difference between the level of the at least one biomarker detected before the treatment and the level in the healthy subject represents that said treatment is effective for the subject.

13. The method according to claim 9, further comprising obtaining information to evaluate response to treatment with a MEK 1/2 inhibitor in the subject with the neurodegenerative disease based on the measured level of the at least one biomarker.

14. The method according to claim 13, wherein the information is used to determine a subsequent dose or duration of treatment with the MEK 1/2 inhibitor, or to determine whether to continue or stop the administration of the MEK 1/2 inhibitor.

15. (canceled)

16. (canceled)

17. A method of treating a subject with a neurodegenerative disease, the method comprising administering a daily dose of a pharmaceutical composition comprising a MEK 1/2 inhibitor in an amount effective to induce change in the level of at least one biomarker selected from the group consisting of osteopontin, synaptotagmin-1, apolipoprotein-E, cathepsin B, HLA class II histocompatibility antigen, DO beta chain (HLA-DOB), and neurofilament light chain in a biological sample from the subject after the administration as compared to prior to administration.

18. A method of determining therapeutic efficacy of a MEK 1/2 inhibitor on a neurodegenerative disease, the method comprising the step of measuring a level of at least one biomarker selected from the group consisting of osteopontin, synaptotagmin-1, apolipoprotein-E, cathepsin B, HLA class II histocompatibility antigen, DO beta chain (HLA-DOB), and neurofilament light chain in a biological sample from a subject with the neurodegenerative disease using a probe specifically binding to the at least one biomarker.
